(19) United States
(12) Patent Application Publication
(10) Pub. No.: US 2017/0165254 A1
(43) Pub. Date: Jun. 15, 2017

(54) TREATING FLAVIVIRUS INFECTIONS WITH AMODIAQUINE AND DERIVATIVES THEREOF

(71) Applicants: Kuppuswamy Nagarajan, Bangalore (IN); GEORGETOWN UNIVERSITY, Washington, DC (US)

(72) Inventors: Kuppuswamy Nagarajan, Bangalore (IN); Radhakrishnan Padmanabhan, Bethesda, MD (US); Siwaporn Boonyasuppayakorn, Bangkok (TH)

(73) Assignee: GEORGETOWN UNIVERSITY, Washington, DC (US)

(21) Appl. No.: 15/116,113

(22) PCT Filed: Feb. 5, 2015
(86) PCT No.: PCT/US15/14578

§ 371 Date: Aug. 2, 2016

Related U.S. Application Data
(60) Provisional application No. 61/936,453, filed on Feb. 6, 2014.

(51) Int. Cl. A61K 31/47; A61K 31/4709; A61K 31/473; A61K 31/357; A61K 45/06

(52) U.S. Cl. CPC A61K 31/47; A61K 31/357; A61K 45/06; A61K 31/473; A61K 31/4709

(57) ABSTRACT

Methods of treating, preventing, and/or ameliorating a Flavivirus infection in a subject are disclosed. The methods comprise administering to the subject a therapeutically effective amount of a Flavivirus inhibitor. These methods are useful in treating, preventing, and/or ameliorating Flavivirus infections such as, for example, West Nile Virus, Dengue Virus, and Japanese Encephalitis Virus.

### CROSS-REFERENCE TO PRIORITY APPLICATIONS

This application claims priority to U.S. Provisional Application No. 61/936,453, filed Feb. 6, 2014, and is incorporated by reference herein in its entirety.

### STATEMENT REGARDING FEDERALLY FUNDED RESEARCH

This invention was made with government support under Grant Nos. R01-AI70791 and U01-AI082068, awarded by the National Institutes of Health. The government has certain rights in the invention.

### BACKGROUND

Flaviviruses such as West Nile virus (WNV), Japanese Encephalitis virus, and Dengue virus (e.g., the four known serotypes of Dengue virus (DENV-1-4)) are significant human pathogens that cause millions of infections each year. Dengue virus (DENV), like other Flaviviruses, has a positive-strand RNA genome. DENV viruses cause a simple and self-limiting disease in humans called dengue fever (DF), which often resolves in a week to 10 days. However, more severe forms of the disease, known as Dengue hemorrhagic fever (DHF) and Dengue shock syndrome (DSS), common in areas endemic to DENV 1-4, lead to considerable morbidity and mortality. According to an estimate reported in 2013, the four serotypes of DENV cause 390 million infections annually. Secondary infections by different DENV serotypes could lead to severe clinical manifestations resulting in approximately 25,000 deaths annually due to antibody dependent enhancement.

WNV was introduced into the western hemisphere during an outbreak in the United States in 1999. In the following years, WNV has spread throughout much of North America and has become a public health concern. Most WNV infections are asymptomatic; however, about 20% of cases are associated with mild flu-like symptoms. A small fraction of these cases progress to more severe clinical manifestations, including encephalitis and/or flaccid paralysis. Currently, there are no approved vaccines or antiviral therapeutics available for either DENV- or WNV-infected humans.

### SUMMARY

Novel methods for treating Flavivirus infections, including the West Nile Virus, Dengue Virus (serotypes DENV-1, DENV-2, DENV-3, and DENV-4), and Japanese Encephalitis Virus, are provided. The methods comprise administering to a subject a therapeutically effective amount of a Flavivirus inhibitor. For example, a method of treating a Flavivirus infection in a subject includes administering to the subject a therapeutically effective amount of a compound of the following formula:

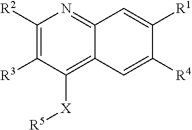

or pharmaceutically acceptable salts or prodrugs thereof. In these compounds, R¹ is hydrogen or halogen; R² and R³ are each independently selected from the group consisting of hydrogen, substituted or unsubstituted alkyl, and substituted or unsubstituted cycloalkyl, wherein optionally R² and R³ combine to form a substituted or unsubstituted aryl, substituted or unsubstituted cycloalkyl, substituted or unsubstituted heteroaryl, or substituted or unsubstituted heterocycloalkyl; R⁴ is hydrogen or substituted or unsubstituted alkoxyl; R⁵ is selected from the group consisting of substituted or unsubstituted alkyl, substituted or unsubstituted heteroalkyl, substituted or unsubstituted cycloalkyl, and substituted or unsubstituted aryl; and X is NH or CH(OH). Optionally, the compound does not include a bis(2-chloroethyl) amine moiety.

Optionally, the compound has the following formula:

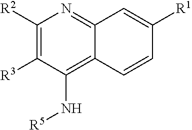

or a pharmaceutically acceptable salt or prodrug thereof.

Optionally, the compound has the following formula:

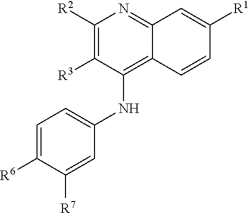

or a pharmaceutically acceptable salt or prodrug thereof. In these compounds, R⁶ and R⁷ are each independently selected from the group consisting of hydrogen, hydroxyl, alkoxyl, and substituted or unsubstituted alkyl.

Optionally, the compound has the following formula:

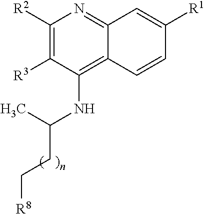

or a pharmaceutically acceptable salt or prodrug thereof. In these compounds, n is 0, 1, 2, or 3; and R⁸ is substituted or unsubstituted amino or substituted or unsubstituted aryl.

Optionally, the compound has the following formula:

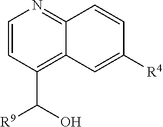

or a pharmaceutically acceptable salt or prodrug thereof. In these compounds, R⁹ is substituted or unsubstituted cycloalkyl or substituted or unsubstituted heterocycloalkyl.

Optionally, the compound has the following formula:

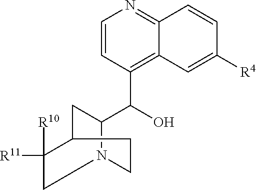

or a pharmaceutically acceptable salt or prodrug thereof. In these compounds, R¹⁰ and R¹¹ are each independently hydrogen or substituted or unsubstituted alkyl, wherein optionally R¹⁰ and R¹¹ combine to form a substituted or unsubstituted alkenyl.

Optionally, the Flavivirus is the West Nile Virus, Dengue Virus serotype DENV-1, Dengue Virus serotype DENV-2, Dengue Virus serotype DENV-3, Dengue Virus serotype DENV-4, or Japanese Encephalitis Virus.

The methods can further comprise administering one or more additional agents to the subject. Optionally, the one or more additional agents include artesunate or a viral protease inhibitor.

The details of one or more embodiments are set forth in the drawings and the description below. Other features, objects, and advantages will be apparent from the description and drawings, and from the claims.

### DESCRIPTION OF DRAWINGS

FIG. 1A and FIG. 1B are graphs showing the percent inhibition of Dengue Virus 2 (DENV2) replicon and WNV replicon by the compounds described herein at 50 μM in 1% DMSO.

FIG. 2A, FIG. 2B, and FIG. 2C are graphs used to determine the EC₅₀ and CC₅₀ values of amodiaquine (AQ) for replicon inhibition and viability of replicon expressing cells, including BHK-21/DENV2 replicon cells (FIG. 2A), Vero/DENV4 replicon cells (FIG. 2B), and Vero/WNV replicon cells (FIG. 2C) (˜10⁴ in 100 μL). Amodiaquine (AQ) was added at concentrations of 0, 0.01, 0.1, 1, 2.5, 5, 7.5, 10, 20, 30, 40, 50, 60, 70, 80, or 100 μM in 1% DMSO.

FIG. 3A is a graph showing the inhibition of DENV2 replication by AQ (5 μM) as analyzed by qPCR and infectivity (plaque) assay.

FIG. 3B is a plot demonstrating the EC₉₀ value determinations for inhibition of DENV2 infectivity by AQ. BHK-21 cells were infected with DENV2 (MOI of 1) and treated with AQ at 0.1, 0.5, 1, 2.5, 5, or 10 μM, or infected with DENV2 (MOI of 0.01) and treated with 0.01, 0.1, 0.5, 0.75, 1, 2.5, 5, 7.5, 10, or 25 μM, during infection and post-infection.

FIG. 4A and FIG. 4B show the time-course analysis results of AQ inhibition of DENV2 infectivity. BHK-21 cells were infected with DENV2 (MOI of 0.01). AQ was added during infection and post-infection at 0, 1, 5, 10, or 25 μM in 1% DMSO or 1% DMSO alone. FIG. 4C is a graph showing the measurement of AQ inhibition of DENV2 infectivity by direct plaque assay using BHK-21 cells and AQ at final concentrations of 0, 1, 5, 10, or 25 μM in 1% DMSO. FIG. 4D is a graph showing the order of addition assay results. AQ and DENV2 (MOI of 1) were treated as follows: 1) AQ and the virus were pre-incubated at 37° C. for 15 minutes before adsorption to BHK-21 cells for 1 hour (preincubation); 2) AQ and the virus were added to BHK-21 cells together and incubated for 1 hour (coinfection); 3) AQ was added after virus adsorption and wash with PBS (postinfection). FIG. 4E is a graph depicting the time of addition assay results from adding AQ (5 μM in 1% DMSO) to DENV2 infected BHK-21 cells (MOI of 1) at 1, 3, 6, 9, 12, 15, 18, 21, 24, 30, 36, or 48 hours post-infection. FIG. 4F is a graph depicting the time of addition assay results from adding AQ (5 μM in 1% DMSO) or 1% DMSO alone to DENV2-infected BHK-21 cells (MOI of 1) at 1, 2, 3, 4, 5, 6, 8, 10, 12, 24, or 48 hours post-infection.

FIG. 5 is a plot demonstrating the EC₉₀ value determinations of AQ, chloroquine (CQ), and AQD8. BHK-21 cells were infected with DENV2 in duplicate wells at a MOI of 1. AQ was added at 0.1, 0.5, 1, 2.5, 5, or 10 μM in 1% DMSO, and CQ and AQD8 were each added at 0.1, 0.5, 1, 2.5, 5, 10, 25, or 50 μM in 1% DMSO.

### DETAILED DESCRIPTION

Methods of treating a Flavivirus infection in a subject comprising administering to the subject a therapeutically effective amount of Flavivirus inhibitors are disclosed. These methods are useful in treating, preventing, and/or ameliorating Flavivirus infections such as, for example, West Nile Virus, Dengue Virus, and Japanese Encephalitis Virus.

I. Compounds

Flavivirus inhibitors useful in the methods described herein comprise compounds represented by Formula I:

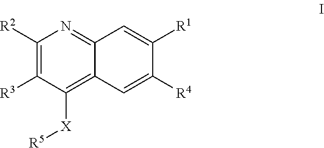

or a pharmaceutically acceptable salt or prodrug thereof.

In Formula I, R¹ is hydrogen or halogen.

Also, in Formula I, R² and R³ are each independently selected from the group consisting of hydrogen, substituted or unsubstituted alkyl, and substituted or unsubstituted cycloalkyl. Optionally, R² and R³ combine to form a substituted or unsubstituted aryl, substituted or unsubstituted cycloalkyl, substituted or unsubstituted heteroaryl, or substituted or unsubstituted heterocycloalkyl.

Additionally, in Formula I, R⁴ is hydrogen or substituted or unsubstituted alkoxyl.

Further, in Formula I, R⁵ is selected from the group consisting of substituted or unsubstituted alkyl, substituted or unsubstituted heteroalkyl, substituted or unsubstituted cycloalkyl, and substituted or unsubstituted aryl.

Additionally, in Formula I, X is NH or CH(OH).

In Formula I, X can be NH to form the following structure represented by Structure A:

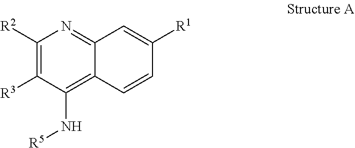

or a pharmaceutically acceptable salt or prodrug thereof. In Structure A, R¹, R², R³, and R⁵ are as defined above in Formula I.

Optionally, in Structure A, R⁵ can be substituted or unsubstituted aryl to form the following structure represented by Structure A-1:

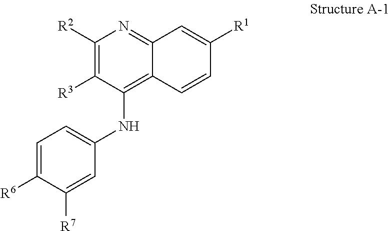

or a pharmaceutically acceptable salt or prodrug thereof. In Structure A-1, R¹, R², and R³ are as defined above in Formula I. Also in Structure A-1, R⁶ and R⁷ are each independently selected from the group consisting of hydrogen, hydroxyl, alkoxyl, and substituted or unsubstituted alkyl. Optionally, R⁶ is hydroxyl or alkoxyl.

Examples of Structure A-1 include the following compounds:

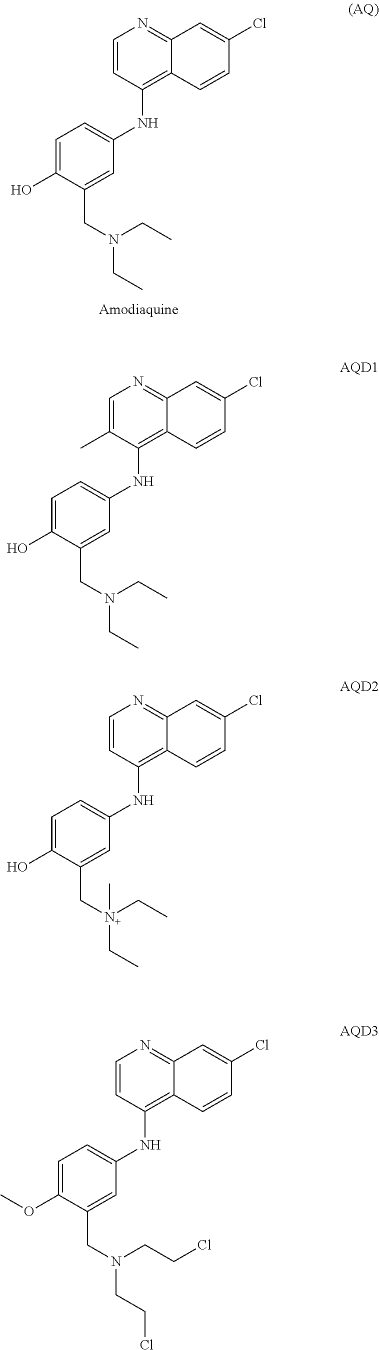 [SMILES: CCN(CC)Cc1cc(Nc2ccnc3cc(Cl)ccc23)ccc1O ; CCN(CC)Cc1cc(Nc2c(C)cnc3cc(Cl)ccc23)ccc1O ; CC[N+](C)(CC)Cc1cc(Nc2ccnc3cc(Cl)ccc23)ccc1O ; COc1ccc(Nc2ccnc3cc(Cl)ccc23)cc1CN(CCCl)CCCl]

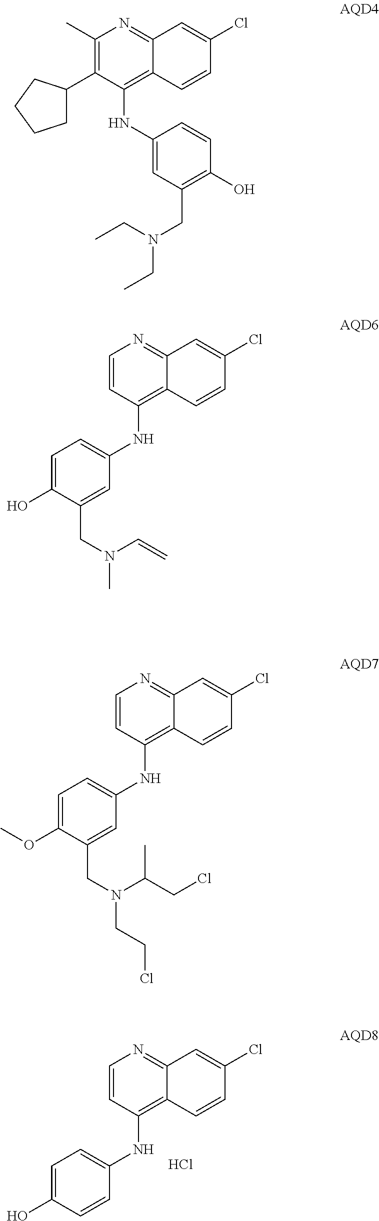 [SMILES: CCN(CC)Cc1cc(Nc2c(C3CCCC3)c(C)nc3cc(Cl)ccc23)ccc1O ; C=CN(C)Cc1cc(Nc2ccnc3cc(Cl)ccc23)ccc1O ; COc1ccc(Nc2ccnc3cc(Cl)ccc23)cc1CN(CCCl)C(C)CCl ; Oc1ccc(Nc2ccnc3cc(Cl)ccc23)cc1 ; Cl]

Optionally, in Structure A, R⁵ can be substituted or unsubstituted alkyl or substituted or unsubstituted heteroalkyl to form the following structure represented by Structure A-2:

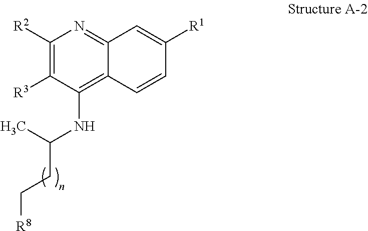

or a pharmaceutically acceptable salt or prodrug thereof. In Structure A-2, R¹, R², and R³ are as defined above in Formula I. Also, in Structure A-2, n is 0, 1, 2, or 3. Additionally, in Structure A-2, R⁸ is substituted or unsubstituted amino or substituted or unsubstituted aryl.

Examples of Structure A-2 include the following compounds:

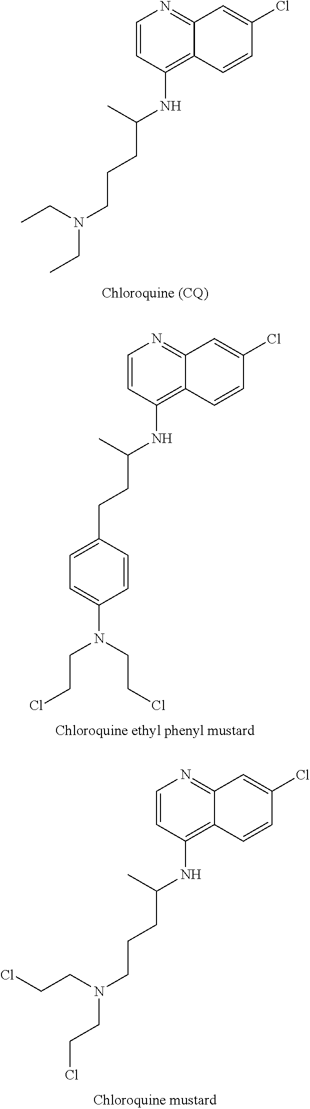 [SMILES: CCN(CC)CCCC(C)Nc1ccnc2cc(Cl)ccc12 ; CC(CCc1ccc(N(CCCl)CCCl)cc1)Nc1ccnc2cc(Cl)ccc12 ; CC(CCCN(CCCl)CCCl)Nc1ccnc2cc(Cl)ccc12]

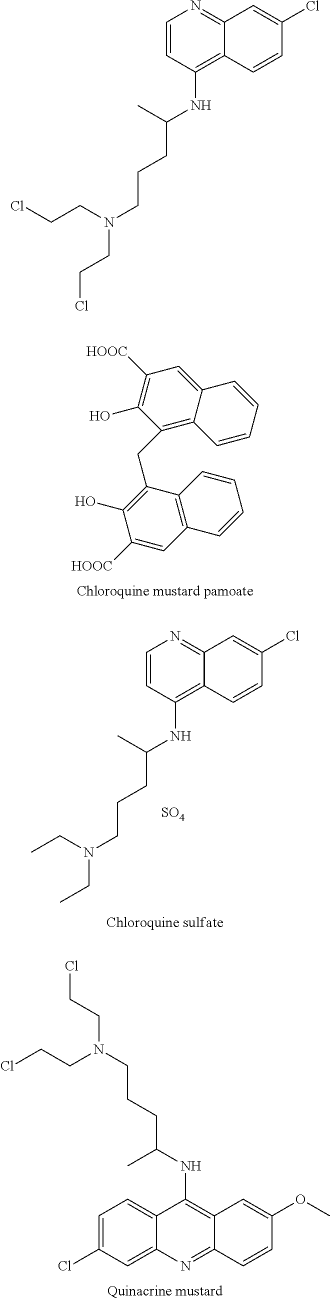

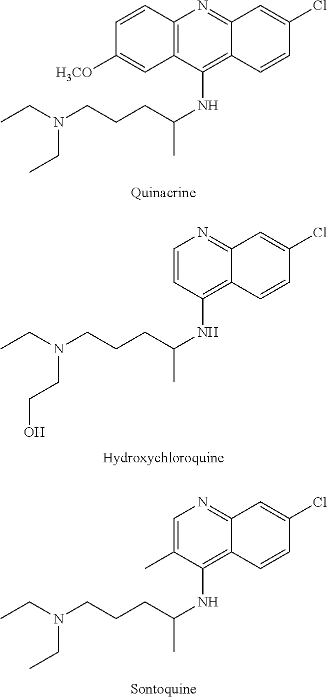 [SMILES: CCN(CC)CCCC(C)Nc1c2ccc(Cl)cc2nc2ccc(OC)cc12 ; CCN(CCO)CCCC(C)Nc1ccnc2cc(Cl)ccc12 ; CCN(CC)CCCC(C)Nc1c(C)cnc2cc(Cl)ccc12]

Optionally, R⁸ does not include a bis(2-chloroethyl) amine moiety (i.e., the compound is not a mustard compound).

In Formula I, X can be CH(OH) to form the following structure represented by Structure B:

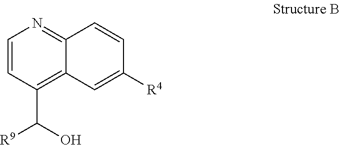

or a pharmaceutically acceptable salt or prodrug thereof. In Structure B, R⁴ is as defined above in Formula I. Also, in Structure B, R⁹ is substituted or unsubstituted cycloalkyl or substituted or unsubstituted heterocycloalkyl.

Optionally, in Structure B, R⁹ can be a bicyclic compound to form the structure represented by Structure B-1:

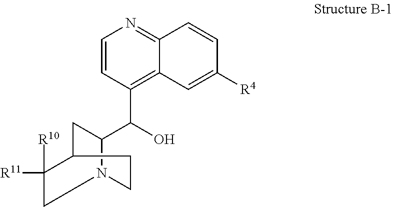

or a pharmaceutically acceptable salt or prodrug thereof. In Structure B-1, R⁴ is as defined above in Formula I. Also in Structure B-1, R¹⁰ and R¹¹ are each independently be hydrogen or substituted or unsubstituted alkyl. Optionally, in Structure B-1, R¹⁰ and R¹¹ can combine to form a substituted or unsubstituted alkenyl.

Examples of Structure B-1 include the following compounds:

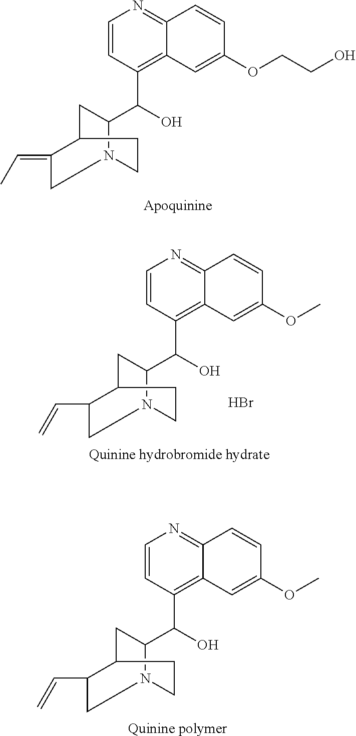

Further examples of compounds for use in the methods described herein can include:

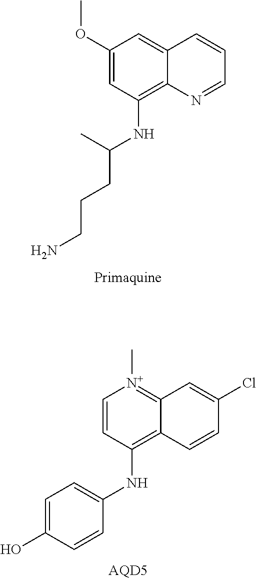

Optionally, the compounds for use in the methods described herein are not chloroquine ethyl phenyl mustard, chloroquine mustard, chloroquine mustard pamoate, quinacrine mustard, primaquine, or quinine polymer. Optionally, the compounds for use in the methods described herein do not include a bis(2-chloroethyl) amine moiety.

As used herein, the terms alkyl, alkenyl, and alkynyl include straight- and branched-chain monovalent substituents. Examples include methyl, ethyl, isobutyl, 3-butynyl, and the like. Ranges of these groups useful with the compounds and methods described herein include C₁-C₂₀ alkyl, C₂-C₂₀ alkenyl, and C₂-C₂₀ alkynyl. Additional ranges of these groups useful with the compounds and methods described herein include C₁-C₁₂ alkyl, C₂-C₁₂ alkenyl, C₂-C₁₂ alkynyl, C₁-C₆ alkyl, C₂-C₆ alkenyl, C₂-C₆ alkynyl, C₁-C₄ alkyl, C₂-C₄ alkenyl, and C₂-C₄ alkynyl.

Heteroalkyl, heteroalkenyl, and heteroalkynyl are defined similarly as alkyl, alkenyl, and alkynyl, but can contain O, S, or N heteroatoms or combinations thereof within the backbone. Ranges of these groups useful with the compounds and methods described herein include C₁-C₂₀ heteroalkyl, C₂-C₂₀ heteroalkenyl, and C₂-C₂₀ heteroalkynyl. Additional ranges of these groups useful with the compounds and methods described herein include C₁-C₁₂ heteroalkyl, C₂-C₁₂ heteroalkenyl, C₂-C₁₂ heteroalkynyl, C₁-C₆ heteroalkyl, C₂-C₆ heteroalkenyl, C₂-C₆ heteroalkynyl, C₁-C₄ heteroalkyl, C₂-C₄ heteroalkenyl, and C₂-C₄ heteroalkynyl.

The terms cycloalkyl, cycloalkenyl, and cycloalkynyl include cyclic alkyl groups having a single cyclic ring or multiple condensed rings. Examples include cyclohexyl, cyclopentylethyl, and adamantanyl. Ranges of these groups useful with the compounds and methods described herein include C₃-C₂₀ cycloalkyl, C₃-C₂₀ cycloalkenyl, and C₃-C₂₀ cycloalkynyl. Additional ranges of these groups useful with the compounds and methods described herein include C₅-C₁₂ cycloalkyl, C₅-C₁₂ cycloalkenyl, C₅-C₁₂ cycloalkynyl, C₅-C₆ cycloalkyl, C₅-C₆ cycloalkenyl, and C₅-C₆ cycloalkynyl.

The terms heterocycloalkyl, heterocycloalkenyl, and heterocycloalkynyl are defined similarly as cycloalkyl, cycloalkenyl, and cycloalkynyl, but can contain O, S, or N heteroatoms or combinations thereof within the cyclic backbone. An example includes quinuclidinyl. Ranges of these groups useful with the compounds and methods described herein include C₃-C₂₀ heterocycloalkyl, C₃-C₂₀ heterocycloalkenyl, and C₃-C₂₀ heterocycloalkynyl. Additional ranges of these groups useful with the compounds and methods described herein include C₅-C₁₂ heterocycloalkyl, C₅-C₁₂ heterocycloalkenyl, C₅-C₁₂ heterocycloalkynyl, C₅-C₆ heterocycloalkyl, C₅-C₆ heterocycloalkenyl, and C₅-C₆ heterocycloalkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imadazole, oxazole, pyridine, and pyrazine. Aryl and heteroaryl molecules can also include additional fused rings, for example, benzofuran, indole, benzothiophene, naphthalene, anthracene, and quinoline. The aryl and heteroaryl molecules can be attached at any position on the ring, unless otherwise noted.

The alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl molecules used herein can be substituted or unsubstituted. As used herein, the term substituted includes the addition of an alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl group to a position attached to the main chain of the alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl, e.g., the replacement of a hydrogen by one of these molecules. Examples of substitution groups include, but are not limited to, hydroxyl, halogen (e.g., F, Br, Cl, or I), and carboxyl groups. Conversely, as used herein, the term unsubstituted indicates the alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl has a full complement of hydrogens, i.e., commensurate with its saturation level, with no substitutions, e.g., linear decane (—(CH₂)₉—CH₃).

II. Pharmaceutical Formulations

The compounds described herein or derivatives thereof can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22^{nd} Edition, ed. Lloyd Allen et al., ed. Pharmaceutical Press (2012). Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN® (ICI, Inc.; Bridgewater, N.J.), polyethylene glycol (PEG), and PLURONICS™ (BASF; Florham Park, N.J.).

Compositions containing the compound described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers, such as cocoa butter, polyethyleneglycol or a suppository wax, which are solid at ordinary temperatures but liquid at body temperature and, therefore, melt in the rectum or vaginal cavity and release the active component.

Dosage forms for topical administration of the compounds described herein or derivatives thereof include ointments, powders, sprays, and inhalants. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

The compositions can include one or more of the compounds described herein and a pharmaceutically acceptable carrier. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught therein.)

Administration of the compounds and compositions described herein or pharmaceutically acceptable salts thereof can be carried out using therapeutically effective amounts of the compounds and compositions described herein or pharmaceutically acceptable salts thereof as described herein for periods of time effective to treat a disorder. The effective amount of the compounds and compositions described herein or pharmaceutically acceptable salts thereof as described herein may be determined by one of ordinary skill in the art and includes exemplary dosage amounts for a mammal of from about 0.5 to about 200 mg/kg of body weight of active compound per day, which may be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

III. Methods of Making the Compounds

The compounds described herein can be prepared in a variety of ways known to one skilled in the art of organic synthesis or variations thereon as appreciated by those skilled in the art. The compounds described herein can be prepared from readily available starting materials. Optimum reaction conditions may vary with the particular reactants or solvents used, but such conditions can be determined by one skilled in the art.

Variations on Formula I and the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

Optionally, one or more of the compounds described herein can be obtained from commercial sources, including, for example, Sigma-Aldrich (St. Louis, Mo.) and other publicly accessible sources, including, for example, the National Cancer Institute's Developmental Therapeutics Program (Bethesda, Md.).

IV. Methods of Use

The methods described above are useful for treating and preventing Flavivirus infections in humans, including, e.g., pediatric and geriatric populations, and animals, e.g., veterinary applications. The methods described herein comprise administering to a subject a therapeutically effective amount of the compounds described herein or a pharmaceutically acceptable salt or prodrug thereof. Flavivirus infections include, for example, West Nile Virus, Dengue Virus, and Japanese Encephalitis Virus. Several serotypes of Dengue Virus have been identified such as, for example, serotype DENV-1, serotype DENV-2, serotype DENV-3, and serotype DENV-4.

The methods described herein are useful for both preventing and treating Flavivirus infections. For prophylactic use, a therapeutically effective amount of the compounds described herein are administered to a subject prior to exposure (e.g., before or when traveling to a location where Flavivirus infections are possible), during a period of potential exposure to Flavivirus infections, or after a period of potential exposure to Flavivirus infections. Prophylactic administration can occur for several days to weeks prior to potential exposure, during a period of potential exposure, and for a period of time, e.g., several days to weeks, after potential exposure. Therapeutic treatment involves administering to a subject a therapeutically effective amount of a compound as described herein after a Flavivirus infection is diagnosed.

In the methods described herein, a Flavivirus infection, for example, can be further treated with one or more additional agents. Optionally, the additional agent can be artemisinin or an artemisinin derivative (e.g., artesunate). Optionally, the additional agent can be a viral protease inhibitor. The one or more additional agents and the compounds described herein or a pharmaceutically acceptable salt or prodrug thereof can be administered in any order, including simultaneous administration, as well as temporally spaced order of up to several days apart. The methods may also include more than a single administration of the one or more additional agents and/or the compounds described herein or a pharmaceutically acceptable salt or prodrug thereof. The administration of the one or more additional agent and the compounds described herein or a pharmaceutically acceptable salt or prodrug thereof may be by the same or different routes and concurrently or sequentially.

V. Kits

Also provided herein are kits for treating or preventing Flavivirus infections in a subject. A kit can include any of the compounds or compositions described herein. For example, a kit can include any of the compounds according to Formula I and other compounds described herein or combinations thereof. A kit can further include one or more additional agents, such as artesunate or a viral protease inhibitor. A kit can additionally include directions for use of the kit (e.g., instructions for treating a Flavivirus infection in a subject), a container, a means for administering the compounds or compositions (e.g., syringe, etc.), and/or a carrier.

As used herein the terms treatment, treat, or treating refer to a method of reducing or delaying one or more symptoms of a Flavivirus infection. Thus in the disclosed method, treatment can refer to a 10%, 20%, 30%, 40%, 50%, 60%, 70%, 80%, 90%, or 100% reduction in the severity or progression of one or more symptoms of the disease or condition. For example, a method for treating a disease is considered to be a treatment if there is a 10% reduction in one or more symptoms or signs of the Flavivirus infection in a subject as compared to a control. As used herein, control refers to the untreated condition. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refer to an action, for example, administration of a composition or therapeutic agent, that occurs before or at about the same time a subject begins to show one or more symptoms of the disease or disorder, which inhibits or delays onset or severity of one or more symptoms of the disease or disorder. For example, the method is considered to be a prevention if there is a reduction or delay in onset, incidence, severity, or recurrence of a Flavivirus infection. The reduction or delay in onset, incidence, severity, or recurrence of a Flavivirus infection can be a 10%, 20%, 30%, 40%, 50%, 60%, 70%, 80%, 90%, 100%, or any percent reduction in between 10% and 100% as compared to native or control levels.

As used herein, references to decreasing, reducing, or inhibiting include a change of 10%, 20%, 30%, 40%, 50%, 60%, 70%, 80%, 90%, or greater as compared to a control level. Such terms can include, but do not necessarily include, complete elimination.

As used herein, subject means both mammals and non-mammals. Mammals include, for example, humans; non-human primates, e.g., apes and monkeys; cattle; horses; sheep; rats; mice; pigs; and goats. Non-mammals include, for example, fish and birds.

Throughout this application, various publications are referenced. The disclosures of these publications in their entireties are hereby incorporated by reference into this application.

The examples below are intended to further illustrate certain aspects of the methods and compounds described herein, and are not intended to limit the scope of the claims.

### Examples

### Example 1: Inhibition of Dengue Virus Type 2 Replication and Infectivity

### Materials and Methods

### Compounds

Amodiaquine dihydrochloride dihydrate (4-[(7-chloroquinolin-4-yl)amino]-2(diethylamino methyl)phenol) (AQ), (Catalog #A2799-5g) was obtained from Sigma Aldrich (St. Louis, Mo.). Quinoline derivatives were obtained from National Cancer Institute/Developmental Therapeutics Program (NCI/DTP) in 10 mg quantities. The compounds were dissolved in DMSO, unless otherwise stated, to prepare 50 mM stock solutions, and were stored as aliquots at −20° C. For some experiments, an aqueous solution of AQ was used as indicated.

### Replicon Inhibition Assay

BHK-21 cells expressing DENV2 replicon (BHK-21/DENV2), Vero cells expressing DENV4 (Vero/DENV4), and WNV (Vero/WNV) replicons were maintained in Dulbecco's Modified Eagle Medium (DMEM) supplemented with 10% fetal bovine serum (FBS), nonessential amino acids (Mediatech, Manassas, Va.), 100 I.U./mL penicillin, 100 μg/mL streptomycin (penicillin-streptomycin), and 300 μg/mL G418 (Fisher Scientific, Pittsburgh, Pa.). Cells (˜10⁴/well) were seeded into 96-well μClear black microtiter plate (Greiner Bio-One, Monroe, N.C.) and were incubated for 6 hours at 37° C. under CO₂ (5%) followed by addition of the compounds in 1% DMSO at final concentrations as indicated. DMSO (1%) alone was used as the no-inhibitor control (100% luciferase activity or 0% inhibition). Cells were incubated at 37° C. for indicated time points. Cells were lysed and Renilla luciferase (Rluc) activities were measured using a Centro LB 960 luminometer (Berthold Technologies, Oak Ridge, Tenn.). Data were reported as percent inhibition relative to 1% DMSO (0% inhibition) and mycophenolic acid (100% inhibition) as controls. Selected compounds showing greater than 80% inhibition were further analyzed to determine the effective concentration at which 50% inhibition was obtained (EC₅₀). To calculate the EC₅₀ values, compounds were serially diluted to final indicated concentrations and the % activity values at various concentrations of the compound were plotted in nonlinear regression using GraphPad Prism v5 software (La Jolla, Calif.).

### Cytotoxicity Assay

Cytotoxicity of AQ was evaluated by two methods. First, naïve BHK-21 or Vero cells were treated with compounds in parallel to the replicon cells. This method was used in evaluating CC₅₀ after 24 hour treatment with the selected compounds. The cell viability was assessed by measuring the ATP level using CellTiter-Glo® luminescent cell viability assay kit (Promega, Madison, Wis.). Briefly, naïve BHK-21 or Vero cells (˜10⁴ cells/well) were seeded in 96-well plates. Cells were incubated for 6 hours at 37° C. Drugs were added at the same concentrations as in the replicon assays. CellTiter-Glo® substrate was added and the plate was read in a luminometer. Data were analyzed to determine the 50% cell viability (CC₅₀) value using GraphPad Prism v5 software.

In the second method, the viability of replicon expressing cells was measured simultaneously using Cell Counting Kit-8 (Dojindo Molecular Technologies, Rockville, Md.) at 2 hours before lysis and Rluc activity measurements. This colorimetric assay utilized highly soluble and non-cytotoxic tetrazolium salt (WST-8), which was added to the experimental cultures and incubated at 37° C. for 2 hours. The plate was read at A_{585 nm} using the Concert TRIAD spectrophotometer (Dynex, Chantilly, Va.). Cells were washed, lysed, and the Rluc activities were measured as described above. CC₅₀ values were calculated using the GraphPad Prism v5 software.

### Inhibition of DENV2 RNA Replication and Infectivity in BHK-21 Cells

BHK-21 cells were seeded into 12-well plates (10⁵ cells/well) and incubated overnight at 37° C. Cells were infected with DENV2 at a multiplicity of infection (MOI) of 0.01 or 1 as indicated. After infection, cells were washed with PBS and incubated with 1.5 mL of MEM supplemented with 2% FBS, 100 I.U./mL penicillin/100 μg/mL streptomycin (referred to as maintenance medium). AQ, at indicated concentrations, was added and cells were incubated at 37° C. for various time points as indicated. DMSO (1%), as a no-compound control (100% infection), and mock-infected control using medium alone (0% infection) were included. Supernatants were collected from the time point experiments and the virus titers determined by plaque assay.

### Plaque Assay

BHK-21 cells were seeded at ˜10⁵ cells/well (12-well plate) or ˜5×10⁴ cells/well (24-well plate) and then incubated at 37° C. until reaching 90% confluence. Cells were infected with the supernatants collected from experiments of AQ treatments. Cells were washed with PBS, and incubated with 1.5 mL of overlay medium (maintenance medium containing 1% methylcellulose). The plates were incubated for 3-4 days at 37° C. under 5% CO₂. After plaques became visually apparent by microscopy, cells were fixed and stained with 11.1% formaldehyde, 4.75% isopropanol, and 1% crystal violet for 30 minutes. The number of plaque forming units (PFU) per mL was determined.

EC₅₀ and EC₉₀ Measurements by Plaque Assay

BHK-21 cells were seeded as described above. AQ, at indicated concentrations in 1% DMSO, was added to DENV2 (MOI of 0.01 or 1)-infected cells during adsorption and/or post-infection. Supernatants were collected at indicated time points post-infection for plaque assays.

Quantitative RT-PCR (qPCR)

Intracellular RNAs were extracted from the infected cells by treatment with TRIzol reagent (Invitrogen, Life, Grand Island, N.Y.). Total RNAs were quantified using Nanodrop 1000 (Thermo Fisher Scientific, Waltham, Mass.) and adjusted to 1 μg/μ1 for reverse transcription (iScript cDNA synthesis, BioRad, Hercules, Calif.). Quantitative RT-PCR (qPCR) was performed. Briefly, the region of viral RNA encoding DENV2 NS1 gene was amplified using the forward and reverse primers (DENV2 NS1-F and DENV2 NS1-R). The glyceraldehyde 3-phosphate dehydrogenase gene (GAPDH) was chosen as the housekeeping reference RNA and amplified by PCR using the forward and reverse primers. The viral RNA copy numbers were calculated in AQ-treated cells relative to the DENV2-infected and AQ-untreated cells (1% DMSO alone). Supernatants from the experiment were also collected and stored as aliquots (1 mL) at −70° C. until use. Supernatant was concentrated by Amicon-15 (Millipore, Billerica, Mass.). Viral RNAs were extracted by QIAamp viral RNA mini kit (QIAgen, Valencia, Calif.) prior to cDNA synthesis as described above.

### Time-Course Analysis on DENV2 Infectivity

BHK-21 cells were grown in 6-well plates (˜2.5×10⁵ cells/well) and were infected with DENV2 at a MOI of 0.01 in the maintenance medium. Cells were washed with PBS and incubated with 3 mL of maintenance medium. AQ at indicated concentrations was added and cells were incubated at 37° C. Supernatants were sampled as indicated and stored at −70° C. for plaque assays.

### Order of Addition Assay

BHK-21 cells were grown in 12-well plates and were treated with AQ in one of three ways: (1) AQ (5 μM) and DENV2 (MOI of 1) were diluted in maintenance medium and incubated for 15 minutes at 37° C. before adding to BHK-21 cells (pre-incubation); (2) the mixture of AQ and DENV2 was added to BHK-21 cells directly (co-infection); or (3) BHK-21 cells were infected with DENV2, or mock-infected with 1% DMSO containing medium first (1 hour at 37° C.), washed, and incubated with medium containing AQ (post-infection). Supernatants were collected at 24, 48, 72 hours post-infection for plaque assays.

### Time of Addition Assay

BHK-21 cells were grown in 12-well plates and infected with DENV2 (MOI of 1). AQ (5 μM) or DMSO alone (1%) was added to DENV2-infected cells at different times post-infection. Supernatants were collected at 72 hours post-infection to determine the titer by plaque assay.

In addition, a time of addition assay was performed to study the effect of AQ (5 μM) on viral translation by collecting more samples as indicated within the first 12 hours after addition of AQ or DMSO (1%) (MOI of 1). Supernatants were collected at 48 hours post-infection for plaque assay.

### Results

### Screening of Antimalarial Compounds for Flaviviral Replication Inhibition

Antimalarial quinoline compounds were screened using BHK-21/DENV2 and Vero/WNV replicon cells (Table 1). Antimalarial compounds that were tested at 50 μM final concentration include 4-aminoquinoline derivatives such as chloroquine (CQ), chloroquine ethyl phenyl mustard, chloroquine mustard, chloroquine pamoate, chloroquine sulfate, amodiaquine (AQ); 6-methoxyquinoline derivatives such as apoquinine, primaquine, and quinine; quinine hydrobromide hydrate; and an acridine derivative, quinacrine mustard. In Table 1, “-” refers to no inhibition.

TABLE 1

| | % Replicon Inhibition (Means ± | |
|---|---|---|
| | SEM) | |
| Name | DENV2 | WNV |
| amodiaquine (AQ) | 76.3 ± 1.6 | 96.3 ± 0.4 |
| apoquinine | — | 33.7 ± 2.9 |
| chloroquine (CQ) | 54.8 ± 4.5 | 18.5 ± 2.8 |
| chloroquine ethyl phenyl mustard | 99.8 ± 0.03 | 99.5 ± 0.05 |
| chloroquine mustard | 99.6 ± 0.32 | 99.8 ± 0.03 |
| chloroquine mustard pamoate | 47.5 ± 4.9 | 10.35 ± 4.1 |
| chloroquine sulfate | 28.1 ± 3.6 | — |
| primaquine | — | — |
| quinacrine mustard | 99.9 ± 0.01 | 99.8 ± 0.0 |
| quinine, hydrobromide hydrate | 2.8 ± 0.25 | 34.9 ± 6.7 |
| quinine, polymers | — | — |
| AQ derivative 1 | 89.9 ± 10.6 | 97.9 ± 0.5 |
| AQ derivative 2 | 69.9 ± 3.4 | 50.6 ± 4.4 |
| AQ derivative 3 | 99.9 ± 0.1 | 99.9 ± 0.02 |
| AQ derivative 4 | 99.99 ± 0.0 | 99.8 ± 0.01 |
| AQ derivative 5 | 20.7 ± 2.9 | 84.5 ± 5.4 |
| AQ derivative 6 | 98.5 ± 1.3 | 99.5 ± 0.1 |
| AQ derivative 7 | 93.0 ± 2.6 | 97.7 ± 1.1 |
| AQ derivative 8 | 3.9 ± 0.3 | 57.2 ± 5.1 |

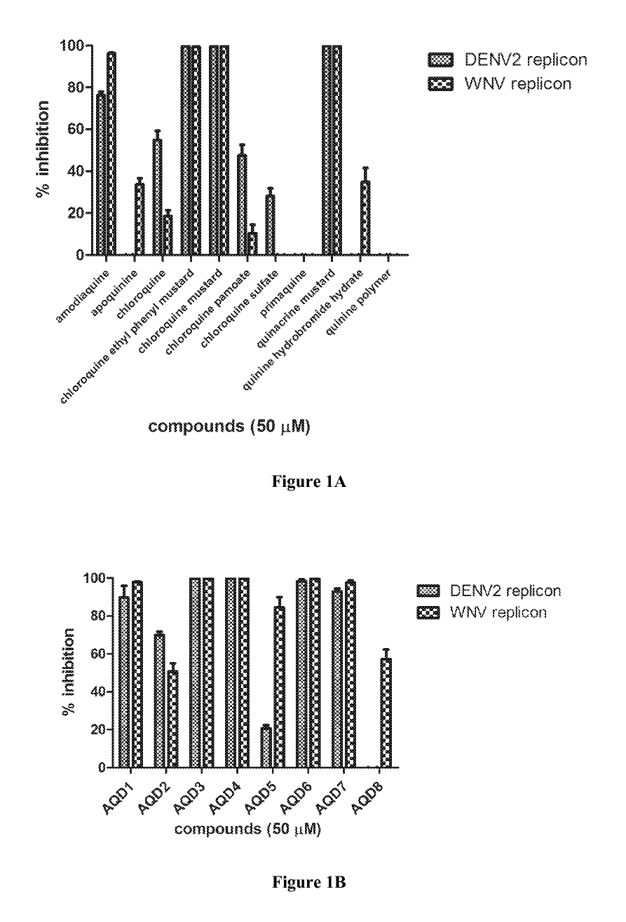

AQ, having a diethylaminomethyl group, showed 76.31±1.60% and 96.30±0.39% inhibition of DENV2 and WNV replicon replication, respectively. Two chloroquine derivatives and one acridine derivative (chloroquine ethyl phenyl mustard, chloroquine mustard, and quinacrine mustard) showed >99% inhibition of both BHK-21/DENV2 and Vero/WNV replicon replication (FIG. 1A). These derivatives contain a common side chain, a dichloroethylamino group, also known as a mustard group. However, chloroquine ethyl phenyl mustard, chloroquine mustard, and quinacrine mustard had low TI values in the range of 2-3 in inhibition of Vero/WNV replicon replication due to their cytotoxicity to Vero cells (Table 2).

TABLE 2

| | DENV replicon | | | WNV replicon | |
|---|---|---|---|---|---|
| Compounds | EC₅₀ | CC₅₀ | TI | EC₅₀ | CC₅₀ |
| AQ | 10.81 ± 1.43 | 80.01 ± 6.27 | 7.4 | 14.63 ± 2.21 | 24.40 ± 2.49 |
| Quinacrine | 0.39 ± 0.10 | 2.40 ± 0.63 | 6.15 | 0.36 ± 0.09 | 0.97 ± 0.11 |
| mustard | | | | | |
| Chloroquine | 2.90 ± 0.78 | 30.47 ± 9.77 | 10.5 | 2.95 ± 0.77 | 3.64 ± 0.93 |
| mustard | | | | | |
| Chloroquine ethyl | 3.60 ± 0.83 | 74.13 ± 21.50 | 20.57 | 1.55 ± 0.40 | 5.02 ± 1.23 |
| phenyl mustard | | | | | |
| AQD1 | 21.09 ± 1.22 | 44.57 ± 2.98 | 2.11 | 14.85 ± 0.48 | 66.37 ± 4.64 |
| AQD2 | 29.68 ± 2.26 | 35.97 ± 5.04 | 1.21 | 30.15 ± 6.05 | 23.15 ± 10.83 |
| AQD3 | 4.76 ± 0.63 | 14.05 ± 2.57 | 2.95 | 8.00 ± 1.27 | 9.36 ± 1.08 |
| AQD4 | 15.36 ± 1.31 | 16.88 ± 2.06 | 1.1 | 3.31 ± 0.73 | 14.58 ± 0.51 |
| AQD5 | 88.71 ± 3.88 | >100 | N/A | 33.40 ± 3.09 | >100 |
| AQD6 | 12.49 ± 2.19 | 16.39 ± 0.46 | 1.31 | 6.29 ± 0.52 | 18.20 ± 1.05 |
| AQD7 | 18.97 ± 1.73 | 17.73 ± 1.96 | 0.93 | 14.84 ± 1.14 | 34.77 ± 1.78 |
| AQD8 | >100 | >100 | N/A | >100 | >100 |

Based on the results of the primary screening (FIG. 1A), eight AQ derivatives (AQD1-8) (Table 1) were further analyzed. AQD1, AQD3, AQD4, AQD6, and AQD7 showed strong inhibition of DENV2 and WNV replicon replication (FIG. 1B).

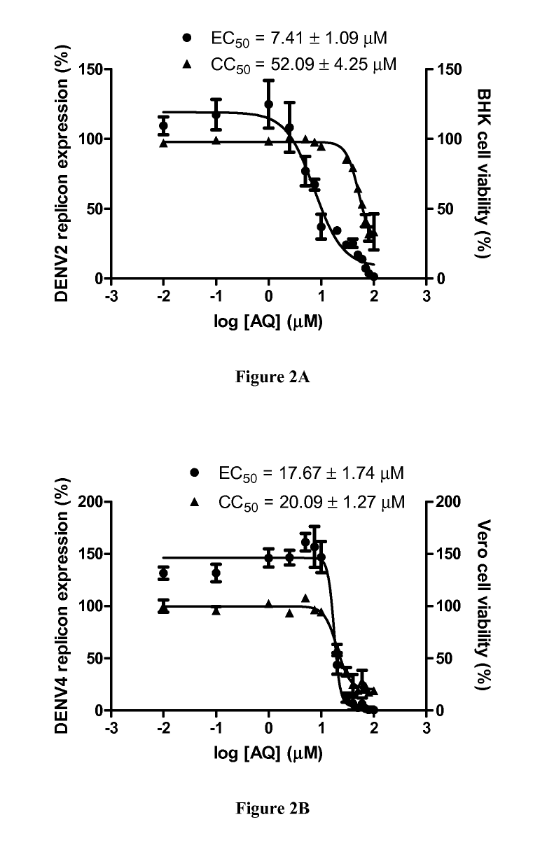

The compounds were tested in BHK-21/DENV2, Vero/DENV4, and Vero/WNV replicon cells and their EC₅₀ values, CC₅₀ values, and therapeutic indices (TIs) were calculated. The TI value of AQ was 7.03 in BHK-21/DENV2 replicon cells, with an EC₅₀ of 7.41±1.09 μM (FIG. 2A), whereas in Vero/DENV4 cells, the TI of AQ was 1.14 (FIG. 2B), and 4.98 in Vero/WNV cells (FIG. 2C) due to the cytotoxicity of AQ to Vero cells. AQ did not interfere with the Rluc enzyme activity when added to the BHK-21/DENV2 replicon cell lysate.

### AQ Inhibits DENV2 Viral RNA Levels and Infectivity

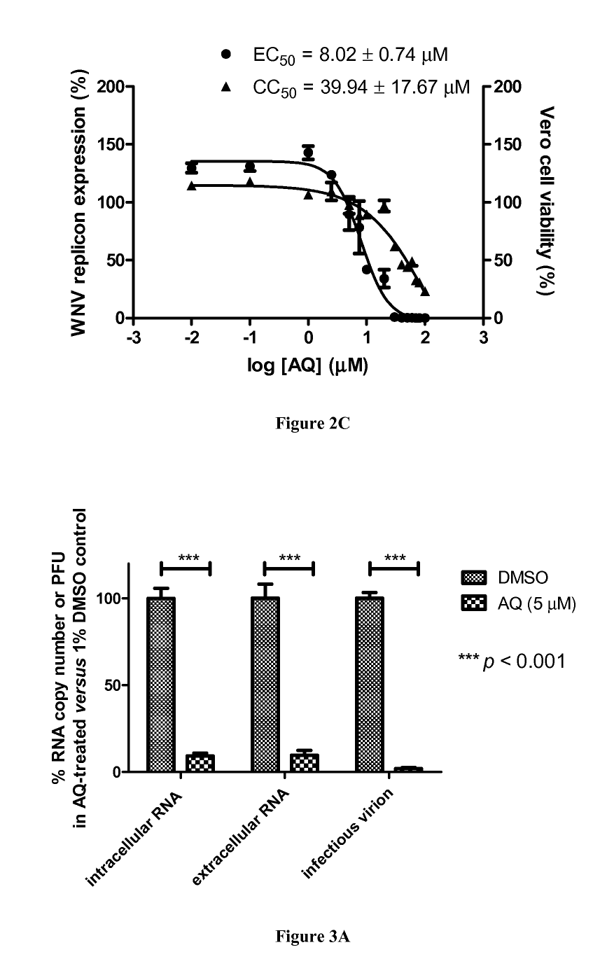

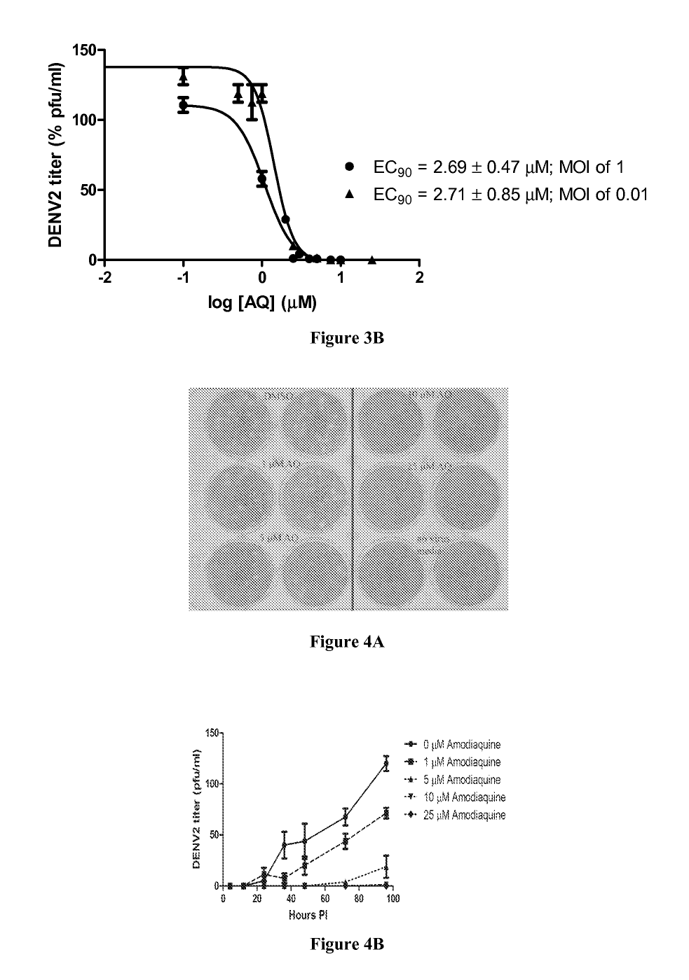

The effect of AQ on the intracellular and extracellular DENV2 RNA levels was analyzed as well as the virus infectivity in BHK-21 cells. The viral replication was quantified by qRT-PCR and the virus infectivity by plaque assay. Cells were treated with a fixed concentration of AQ (5 μM) and the infected cells were incubated for 72 hours (FIG. 3A). Results showed a significant difference (p<0.001) between the AQ-treated and untreated groups. The results indicate that AQ effectively inhibited DENV2 replication with the reducing levels of intracellular and extracellular RNAs. The virus infectivity measured as PFU/mL of supernatant from the AQ-untreated and AQ (5 μM)-treated cells by plaque assay also showed a significant reduction upon treatment with the compound (FIG. 3A).

The EC₅₀ and EC₉₀ values for AQ-mediated inhibition of extracellular release of DENV2 from the infected BHK-21 cells were determined. BHK-21 cells were infected with DENV2 and treated with AQ at various concentrations. The supernatants collected at 72 hours (MOI of 1) or 96 hours (MOI of 0.01) post-infection were analyzed by plaque assay. The virus titers at various concentrations of AQ were plotted using GraphPad Prism v5 software. The EC₅₀ value of 1.08±0.09 μM (MOI of 1) and EC₉₀ value of 2.69±0.40 μM (MOI of 1) as well as EC₉₀ of 2.71±0.85 (MOI of 0.01) from an independent experiment are shown (FIG. 3B).

### Mode of Inhibition of DENV2 Infection by AQ

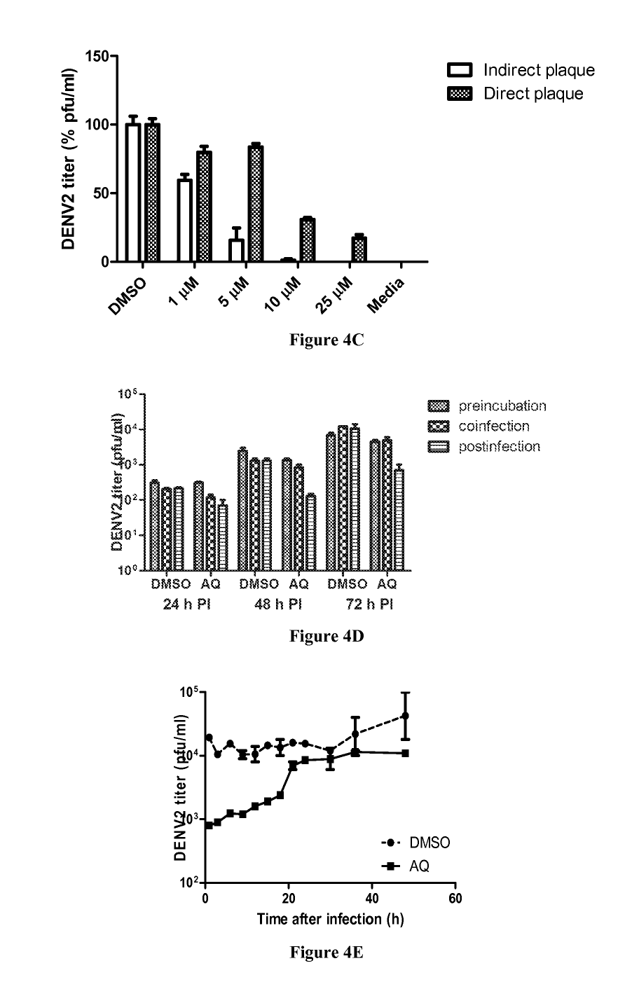

Experiments were performed in BHK-21 cells infected with DENV2 to determine the stage of the virus life cycle targeted by AQ. First, a time course of DENV2 infectivity in the absence and presence of different concentrations of AQ was performed (FIGS. 4A and 4B). AQ was added to the DENV2 infected cells during adsorption and post-infection. As shown in FIGS. 4A and 4B, in mock-infected control cells (1% DMSO alone), DENV2 infectivity titer gradually increased over time. However, in the AQ-treated cells, the virus titers (PFU/mL) were reduced significantly in a dose-dependent manner (p<0.05) (FIGS. 4A and 4B). At 5 μM, the plaque formation was reduced ≧90%. The AQ-mediated inhibition increased over time indicating that AQ was stable at least up to 96 hours post-infection.

To analyze AQ's effect on viral entry, AQ at various concentrations was incubated with DENV2 (MOI of 0.01) on the BHK-21 monolayer for 1 hour adsorption period, and then overlay medium in the absence of drug, was added and incubated at 37° C. for 3-4 days as described above under Materials and Methods. Data obtained from this experiment are labeled in FIG. 4C as direct plaque assay (FIG. 4C, dotted bars). Concurrently, virus supernatants collected from cells infected with DENV2 in the presence of 1% DMSO or AQ present during and up to 96 hours post-infection were also used for plaque assay (referred to in FIG. 4C as indirect plaque assay). The virus titers were determined as above (FIG. 4C, clear bars). The virus titers from indirect plaque assays (FIG. 4C, clear bars) showed significant inhibition of DENV2 infectivity by AQ at 5 μM. However, in the direct plaque assay, since AQ was present only during adsorption, the plaque titer at 5 μM AQ was not significantly different from its DMSO control (FIG. 4C, dotted bars). These results indicate that AQ at 5 μM does not inhibit DENV2 adsorption. However, the virus titer was reduced by 69.23±1.57% and 82.70±2.48% when treated with AQ during adsorption at 10 and 25 μM, respectively, during adsorption (FIG. 4C, dotted bars). The results indicate that the inhibition of DENV2 entry requires a higher concentration of AQ (10 or 25 μM) than that required to inhibit infectivity when added post-infection (5 μM).

To confirm that AQ had the optimal inhibitory effect on DENV2 replication when added during post-infection period, the order of addition of AQ was expanded at various time points as follows. First, a mixture of AQ and DENV2 (MOI of 1) was incubated for 15 minutes at 37° C. prior to addition to BHK-21 cells. Second, AQ and DENV2 (MOI of 1) were simultaneously added to BHK-21 cells. Third, AQ was added to BHK-21 cells post-adsorption with DENV2. Supernatants were collected at 24, 48, and 72 hours post-infection for plaque assay (FIG. 4D). Results indicated that the inhibition of DENV2 infectivity by AQ was maximum (≧90% reduction of plaque titer from its DMSO control) when the drug was added post-infection at 48 and 72 hours (FIG. 4D, striped bar).

To pinpoint the post-infection stage of inhibition more precisely, AQ (5 μM) was added to DENV2 infected BHK-21 cells (MOI of 1) at various time points post-infection. Supernatants were collected at 72 hours post-infection and were analyzed for time-dependent plaque reduction. The plaque reduction by ≧90% was found even when the drug was added as late as 15 hours post-infection (FIG. 4E). At 21 hours post-infection and later time points, the drug lost its inhibitory effect.

The effect of AQ within the first 12 hours post-infection was examined to determine whether AQ is a translational inhibitor similar to a compound with benzomorphan core structure. Results indicated that addition of AQ up to 12 hours inhibited DENV2 infection steadily (FIG. 4F) and even added at 15 hours post-infection (FIG. 4E) beyond which there was a steady increase in titer (loss of inhibition). Thus, AQ can inhibit early events, including translation of viral RNA templates released from the endosomes, and events prior to assembly of viral RNA replicase complex and on-set of viral RNA replication (FIGS. 4, E and F).

### Effect of Diethylaminomethyl Group Adjacent to Phenolic OH of AQ on Inhibition of DENV2 Replication and Infection

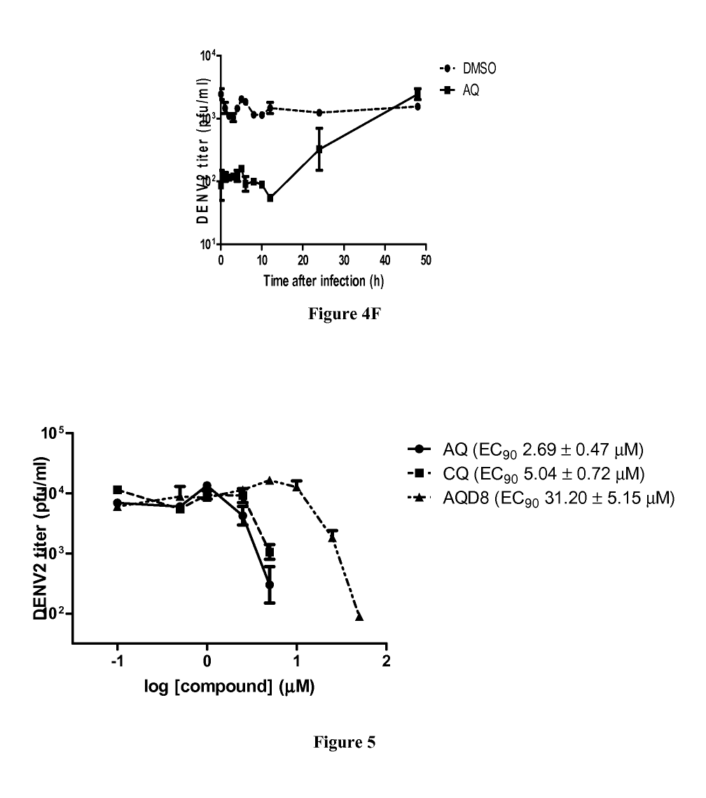

From the analysis of AQ and its derivatives using BHK-21/DENV2 replicon cells, compounds lacking a diethylaminomethyl group, AQD5 and AQD8, failed to inhibit replicon replication. The EC₅₀ values were ˜100 μM (Table 1 and Table 2), at least 10-fold higher than that of AQ. AQD8 was chosen for further study using the infectivity assay. AQD8, at various concentrations, was added to DENV2 infected BHK-21 cells (MOI of 1). After 72 hours incubation, supernatants were collected and the virus titers were determined by plaque assay. Results showed an EC₉₀ of AQD8 at 31.20±5.15 μM (FIG. 5A), which is more than 10 fold higher compared to that of AQ (EC₉₀ of 2.69±0.47 μM). Thus, AQD8 has a modest potency in inhibition of replicon replication and viral infectivity. These results indicate that the diethylaminomethyl group is a structural moiety of AQ that contributes to inhibition of DENV2 replication and infectivity (FIG. 5, B-D).

The effect of CQ, another FDA-approved antimalarial drug and a 4-aminoquinoline derivative, on the replication of DENV2 was determined. BHK-21 cells were infected with DENV2 at a MOI of 1 and treated with CQ at various concentrations. The virus titers of the supernatants were determined by plaque assay. As shown in FIG. 5A, CQ inhibited DENV2 replication in BHK-21 cells in a dose-dependent manner (EC₉₀=5.04±0.72 μM) although it did not inhibit DENV2 replicon replication (FIG. 1).

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods are intended to fall within the scope of the appended claims. Thus a combination of steps, elements, components, or constituents may be explicitly mentioned herein; however, all other combinations of steps, elements, components, and constituents are included, even though not explicitly stated.

1. A method of treating a Flavivirus infection in a subject, comprising:
administering to the subject a therapeutically effective amount of a compound of the following formula: 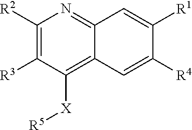
or a pharmaceutically acceptable salt or prodrug thereof, wherein:
R¹ is hydrogen or halogen;
R² and R³ are each independently selected from the group consisting of hydrogen, substituted or unsubstituted alkyl, and substituted or unsubstituted cycloalkyl, wherein optionally R² and R³ combine to form a substituted or unsubstituted aryl, substituted or unsubstituted cycloalkyl, substituted or unsubstituted heteroaryl, or substituted or unsubstituted heterocycloalkyl;
R⁴ is hydrogen or substituted or unsubstituted alkoxyl;
R⁵ is selected from the group consisting of substituted or unsubstituted alkyl, substituted or unsubstituted heteroalkyl, substituted or unsubstituted cycloalkyl, and substituted or unsubstituted aryl; and
X is NH or CH(OH),
wherein the compound does not include a bis(2-chloroethyl) amine moiety.

2. The method of claim 1, wherein the compound has the following formula: 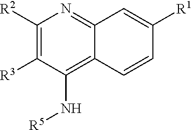
or a pharmaceutically acceptable salt or prodrug thereof.

3. The method of claim 1, wherein the compound has the following formula: 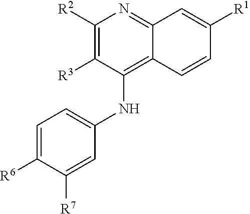
or a pharmaceutically acceptable salt or prodrug thereof, wherein:
R⁶ and R⁷ are each independently selected from the group consisting of hydrogen, hydroxyl, alkoxyl, and substituted or unsubstituted alkyl.

4. The method of claim 1, wherein the compound has the following formula: 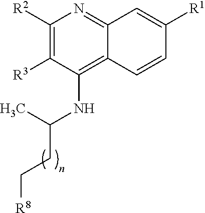
or a pharmaceutically acceptable salt or prodrug thereof, wherein:
n is 0, 1, 2, or 3; and
R⁸ is substituted or unsubstituted amino or substituted or unsubstituted aryl.

5. The method of claim 1, wherein the compound has the following formula: 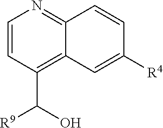
or a pharmaceutically acceptable salt or prodrug thereof, wherein:
R⁹ is substituted or unsubstituted cycloalkyl or substituted or unsubstituted heterocycloalkyl.

6. The method of claim 1, wherein the compound has the following formula: 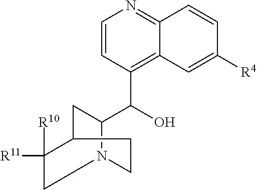
or a pharmaceutically acceptable salt or prodrug thereof, wherein:
R¹⁰ and R¹¹ are each independently hydrogen or substituted or unsubstituted alkyl, wherein optionally R¹⁰ and R¹¹ combine to form a substituted or unsubstituted alkenyl.

7. The method of claim 1, wherein the Flavivirus is the West Nile Virus.

8. The method of claim 1, wherein the Flavivirus is Dengue Virus serotype DENV-1.

9. The method of claim 1, wherein the Flavivirus is Dengue Virus serotype DENV-2.

10. The method of claim 1, wherein the Flavivirus is Dengue Virus serotype DENV-3.

11. The method of claim 1, wherein the Flavivirus is Dengue Virus serotype DENV-4.

12. The method of claim 1, wherein the Flavivirus is Japanese Encephalitis Virus.

13. The method of claim 1, further comprising administering one or more additional agents to the subject.

14. The method of claim 13, wherein the one or more additional agents include artesunate.

15. The method of claim 13, wherein the one or more additional agents includes a viral protease inhibitor.

16. The method of claim 3, wherein:
R¹ is a halogen;
R² and R³ are each hydrogen;
R⁶ is substituted or unsubstituted alkyl; and
R⁷ is hydroxyl.

17. The method of claim 16, wherein R¹ is chloro.

18. The method of claim 16, wherein R⁶ is substituted alkyl.

19. The method of claim 18, wherein the substituted alkyl is an amino-substituted alkyl.

20. A method of treating a Flavivirus infection in a subject, comprising:
administering to the subject a therapeutically effective amount of a compound of the following formula: 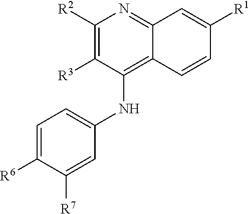
or a pharmaceutically acceptable salt or prodrug thereof, wherein:
R¹ is chloro;
R² and R³ are each hydrogen;
R⁶ is substituted alkyl; and
R⁷ is hydroxyl.

21. The method of claim 20, wherein R⁶ is an amino-substituted alkyl.
